(19) United States
(12) Patent Application Publication
(10) Pub. No.: US 2008/0102438 A1
(43) Pub. Date: May 1, 2008

(54) Novel Technique to Fabricate Molded Structures Having a Patterned Porosity

(76) Inventors: Ioannis V. Yannas, Newton, MA (US); Brendan Harley, Cambridge, MA (US); Abel Z. Hastings, Durham, NC (US); Alessandro Sannino, Lecce (IT)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/666,594

(22) PCT Filed: Oct. 27, 2005
(86) PCT No.: PCT/US05/39024

§ 371 Date: Nov. 9, 2007

Related U.S. Application Data
(60) Provisional application No. 60/622,441, filed on Oct. 27, 2004.

(51) Int. Cl. A01N 1/00; C12N 5/06

(52) U.S. Cl. 435/1.1; 435/383; 264/28; 428/304.4

(57) ABSTRACT

The present invention relates to a process for fabricating molded structures having a radially organized pore structure. The molded structures are formed using a spinning-induced sedimentation technique such that sedimentation of a multi-component liquid suspension produces the internal geometry and porosity of the structure. The porous molded structures of the invention can be used in a number of applications including tissue and organ engineering, dialysis and phase separation membranes and water and liquid waste purification systems.

### RELATED APPLICATION

This application claims the benefit of U.S. Provisional Application No. 60/622,441, filed Oct. 27, 2004. The entire teachings of the above application are incorporated herein by reference.

### GOVERNMENT SUPPORT

The invention was supported, in whole or in part, by a grant, NIH Contract No. 6732300, OSP Project No. DE13053, from the National Institutes of Health. The Government has certain rights in the invention.

### BACKGROUND OF THE INVENTION

In various fields there is a need for porous structures which can be used in membranes for phase separation, tubes for dialysis and devices for water purification and liquid waste treatment. In some applications, it is advantageous for the porous structures to be fabricated with a controlled, radially aligned pore structure. For instance, in tissue engineering and organ regeneration, radially aligned pore channels are able to influence cell behavior, including the direction, prevention and induction of cell growth through the scaffold.

Radially aligned pore structures are thought to significantly improve the quality of regeneration of a variety of tissues. For example, porous tubes have been used extensively in studies of induced peripheral nerve regeneration. Tublation, a process in which the transected stumps of an injured nerve are inserted into either end of a tubular template, profoundly affects the healing process of an injured nerve and is required for the regeneration of a functional nerve after injury. In the past, the quality of nerve regeneration has been enhanced with respect to controls by the proper choice of tube parameters such as conduit diameter and length, lumenal surface microgeometry, and wall porosity and permeability.

Fabrication of structures with either solid or porous walls has been accomplished using a variety of techniques. However, these methods have several drawbacks including limitations in the dimensions of the molds, limitations on pore size and number, the need to use a complex tubular mold and an undesirable complexity in many of the processes. For instance, the use of complex molds requires a careful handling of the product during all stages of fabrication and, even then, the removal of samples from the mold can damage the final product. Further, none of the techniques allow for the production of tubular structures with porous walls aligned into radial channels, a geometry advantageous for tissue engineering. There is a need, therefore, for an ability to manufacture such porous structures through a simple process that carefully controls geometry and porosity of the structures.

### SUMMARY OF THE INVENTION

The present invention relates to a new method for fabricating a porous molded structure comprising spinning a liquid suspension having two or more components of differing densities around a mold axis, immobilizing the two or more components to the mold and removing at least one of the two or more component phases from the bulk, wherein radially aligned pores structures are formed in the molded structure. In one embodiment, the mold geometry is cylindrical. In another embodiment of the method, the density of the two or more components of the liquid suspension, the consistency of the liquid suspension, the spin time and the spin velocity are modulated to produce the internal geometry of the porous tubular structure. In another embodiment, the porous structure has a central lumen that is either hollow or filled and, in a particular embodiment, the porous structure is a tube. In yet another embodiment of the method, the density of the two or more components of the liquid suspension, the consistency of the liquid suspension, the spin time, the spin velocity and the immobilization technique are modulated to produce the internal pore structure of the porous molded structure. In a further embodiment, the porous structure can also be further stabilized.

The present invention also relates to a method for engineering a tissue comprising fabricating a porous molded structure having radially aligned pore channels and growing cells on the structure along the radially aligned pore channels. In one embodiment, the geometry of the molded structure is cylindrical. In another embodiment neuronal cells, fibroblasts, epithelial cells, endothelial cells, epidermal cells, islets of Langerhan cells, osteocytes, tenocytes, chondrocytes, adult stem cells, embryonic stem cells, fetal stem cells or progenitor cells are grown on the radially aligned pore structure of the molded structure. In a further embodiment, the porous cylindrical structure is a tube having a cell-impermeable membrane on either the inner or outer surface of the tube wall that controls the migration of the cells grown on the tubular structure. In another embodiment of the method, neuronal cells are grown on the structure and the tissue engineered is a neural tube. In yet another embodiment, arterial or venous endothelial cells and vascular smooth muscle cells are grown on the structure and the tissue engineered is a blood vessel.

The present invention further relates to a porous molded structure comprising radially aligned pore channels formed by a centrifugation technique. In one embodiment, the porous molded structure is formed by the claimed method. In another embodiment, the porous molded structure is used in tissue engineering, organ engineering, membranes for phase separation, dialysis tubes, water purification systems and liquid waste purification systems.

The present invention further relates to an engineered tissue or organ comprising a molded structure formed from a biocompatible material, said structure seeded with cells grown along a radially aligned pore structure formed by a centrifugation technique, which is. In one embodiment, the molded structure comprising the engineered tissue is seeded with cells selected from the group consisting of neuronal cells, fibroblasts, epithelial cells, endothelial cells, epidermal cells, islets of Langerhan cells, osteocytes, tenocytes, chondrocytes, adult stem cells, embryonic stem cells, fetal stem cells and progenitor cells. In another embodiment, the molded structure comprising the engineered tissue is tubular. In a further embodiment, the cells seeded and grown on the radially aligned pore structure of the molded structure are neuronal cells such that the tissue engineered is a neural tube. In yet another embodiment, the cells seeded on the radially aligned pore structure of the molded structure are arterial or venous endothelial cells and vascular smooth muscle cells and the tissue engineered is a blood vessel.

The methods of the invention provide for a molded structure having radially aligned pores by using a rotation or centrifugation technique that renders a complex mold unnecessary. In lieu of a complex mold, the method relies on the spinning and sedimentation of the components of the liquid suspension to produce the appropriate internal geometry and porosity of the structure.

### BRIEF DESCRIPTION OF THE DRAWINGS

The foregoing and other objects, features and advantages of the invention will be apparent from the following more particular description of preferred embodiments of the invention, as illustrated in the accompanying drawings. The drawings are not necessarily to scale, emphasis instead being placed upon illustrating the principles of the invention.

FIG. 1 is a schematic illustrating the experimental set up for fabrication of a cylindrical tubular structure.

FIG. 2 is a photograph illustrating the pore structure of a collagen-GAG scaffold fabricated by spinning at 30,000 rpm for 15 minutes using the apparatus of FIG. 1.

FIG. 3 is a photograph illustrating the pore structure of a collagen-GAG scaffold fabricated by spinning at 5,000 rpm for 5 minutes using the apparatus of FIG. 1.

FIG. 4 is a photograph illustrating the pore structure of a collagen-GAG scaffold fabricated by spinning at 5,000 rpm for 1 minute using the apparatus of FIG. 1.

### DETAILED DESCRIPTION OF THE INVENTION

The present invention relates to a process for fabricating molded structures (e.g., cylindrical and/or tubular structures) with an organized pore structure using a rotation or centrifugation method. A multi-component liquid suspension forms its own porous template based on the rotational forces imparted on the suspension. A single mold can be used to produce porous structures having a variety of internal diameters by modification of the rate or time of spinning. The pores are organized such that they are aligned radially in the structure, making highly specific porous channels through the structure walls. Structures having pore organization of this type can be used in a variety of industrial applications including tissue and/or organ engineering, dialysis and phase separation membranes and the purification of water and/or liquid waste.

Accordingly, the invention relates to a method for fabricating a porous molded structure comprising spinning a liquid suspension having two or more components of differing densities around a mold axis, immobilizing the two or more components to the mold and removing at least one of the two or more component phases from the bulk such that radially aligned pore structures are formed in the molded structure. The components for use in the liquid suspension can be any two or more components appropriate for the formation of a porous structure provided that the two or more components chosen are of different densities (e.g. collagen and chondroitin or poly-L-lactide and polylactide co-glycolide).

For example, a liquid suspension of two or more components (A+B) is spun in a cylindrical mold around the axis of the cylinder, causing sedimentation. After sedimentation begins, sedimented component phase(s) (A) is immobilized in such a maimer that a radially aligned structure of the component phase(s) is formed. A removal technique is utilized to remove the remaining component phase(s) (B), leaving a porous tube with a radially aligned pore structure with walls produced from component phase(s) (A) surrounding pores in the tube wall and the tube lumen formed by the removal of component phase(s) (B). In the case of the formation of cylindrical rather than tubular structures, the same removal technique results in a porous cylindrical structure with a radially aligned pore structure with walls produced from component phase(s) (A) surrounding pores formed by the removal of component phase(s) (B).

The components can be, for example, collagen (synthetic or animal-derived), glycosaminoglycans (e.g., chondroitin 6-sulfate, keratan sulfate, dermatan sulfate, heparan sulfate, chitin or chitosan), polyphosphoesters, gelatin, fibronectin, laminin, hyaluronic acid, pectin, cellulose derivatives, biodegradable synthetic polymers (e.g., aliphatic polyesters, polyamine acid, polyanhydride, polycaprolactone or polyglycolide) or polylactide derivatives (e.g., lactide and glycolide copolymers) among others. Examples of components and the preparation of components for use in the method of the invention can be found in Yannas et al. U.S. Pat. No. 4,060,081, Yannas et al. U.S. Pat. No. 4,947,840, Yannas et al. U.S. Pat. No. 4,522,753 and Yannas et al. U.S. Pat. No. 4,350,629, the contents of which are herein incorporated by reference. The components can be suspended in a fluid (e.g., a liquid) and the suspension fluid can be any suitable carrier for the chosen components. In a particular embodiment, the two components are suspended in acetic acid. The consistency and density of the multi-component liquid suspension can be varied to produce a specific porous structure, necessary for the particular use of the structure, as determined by one with skill in the art.

A mold is employed to define the shape of the outer surface of the porous structure. The mold can be of a variety of shapes and sizes based on the desired and/or suitable structure for a particular application. The mold can be comprised of a material suitable for the spinning, immobilization and removal steps of the method for the chosen components suspended in a particular carrier (e.g., collagen and chondroitin in acetic acid). The mold material (e.g., oxygen-free copper) can be chosen based on several characteristics including thermal properties (i.e., rapid heat and/or cold conduction), tensile strength, fractionation properties, an ability to tolerate the application of the chosen components and an inability to react with the chosen components, along with other characteristics. In one embodiment, the mold geometry is that of a cylindrical shape.

In the method of the invention, the multi-component liquid suspension is spun around the axis of the chosen mold, causing sedimentation of the suspended components. Sedimentation of the component phases ultimately produces the desired inner geometry of the structure. Due to the different densities of the two or more components, the spinning procedure produces a non-homogenous distribution of the components. Depending on the relative densities and rheological properties of the two or more components, it is possible to produce specific amounts of interpenetration and phase separation by modulating the time and velocity of rotation. Production of a porous material, as in the present invention, occurs due to the interpenetration of the two or more components at the end of the centrifugation process.

Further, the spin velocity of the liquid suspension regulates the internal diameter of the structure. For example, by spinning the mold at a high rate for long times, a large amount of sedimentation will take place at the outer surface of the bulk, resulting in the formation of tubular structure having an impermeable membrane around the outer edge of the tube and a radial gradient in pore size (decreasing toward the outer tube wall) through the tube wall. In addition, at high rotation velocities or long spin times, the two or more components of sufficiently differing densities will separate such that one component is completely removed from the central portion of the mold, resulting in the production of a hollow tube. Advantageously, formation of a porous tube without a central lumen by centrifugation does not require the use of an internal mandrel.

Thus, in an embodiment of the method, the density of the two or more components of the liquid suspension, the consistency of the liquid suspension, the spin time and spin velocity can all be modulated to produce the internal geometry and porosity of the structure.

After sedimentation, the two or more components are immobilized to the bull in a maimer such that a radially aligned pore structure is formed. The immobilization can be accomplished by a variety of techniques (e.g., freezing via immersion of the entire mold in liquid nitrogen) and is generally specific not to the spinning procedure, but to the chosen component materials themselves. Thus, in another embodiment of the method, the immobilization technique along with the density of the two or more components of the liquid suspension, the consistency of the liquid suspension, the spin time and spin velocity are modulated to produce the internal pore structure of the porous molded structure.

At least one of the immobilized component phases is then removed from the sedimented bulk to impart the final inner geometry of the structure, with the porous molded structure retaining a material distribution of the removed component. A number of techniques can be used in the process for removal of the desired component phase(s), a process which is, again, specific to the components themselves. One example of a process for removing at least one of the two or more component phases is a lyophilization (i.e., freeze-drying) technique in which an aqueous solution is removed from a multi-component system by freezing the liquid suspension during the spinning process. The frozen aqueous phase of the solution can then be removed from the bulk by sublimation. Other techniques that can also be utilized to remove the component phase(s) include enzymatic digestion, leaching through the use of a diffusion gradient (e.g., salt), phase inversion and thermal degradation. Both the immobilization and removal aspects of the invention can be tailored to the particular multi-component liquid suspension depending upon the thermodynamic and rheological characteristics of the suspension.

If desired and/or necessary, the porous pattern of the molded structure can be further stabilized by a number of treatments, the treatment being dependent on the characteristics of the components used. For example, in the instance that one of the components is collagen, dehydrothermal treatment (i.e., the reduction of the moisture level of the components) can be used to induce physical crosslinks between the fibers of collagen in the tube, further stabilizing the structure. Crosslinking of the porous structure, depending on the components, could also be achieved by chemical (e.g., carbodiimide and azide coupling or diisocyanate crosslinking), radiation or covalent crosslinking techniques (e.g., aldehyde crosslinking). (See also Yannas et al. U.S. Pat. No. 4,280,954, herein incorporated by reference).

Overall, the selection of the mold for the structure determines the shape of the outer surface of the structure (e.g., cylindrical), while modulation of the multi-component liquid suspension consistency and density, the time of spinning, the speed of spinning and the immobilization technique defines the inner geometry and pore structure of the molded structure. Varying these aspects in the method allows for the production of molded materials having radially aligned pore structures, with or without a central lumen, and having walls of various thicknesses and sizes.

The present invention also relates to a method for engineering a tissue comprising fabricating a porous molded structure having radially aligned pore channels and growing cells on the molded structure along the radially aligned pore channels. Although cells are grown along the radially aligned pore structures in the method, cells can also be grown axially anywhere within the structure, as desired by one with skill in the art. There exist a number of methods for growing cells on a matrix or scaffold and any method known to one of skill in the art for culturing and/or growing cells (e.g., neuronal cells, fibroblasts, epithelial cells, endothelial cells, epidermal cells, islets of Langerhan cells, osteocytes, tenocytes or chondrocytes) obtained from a tissue of interest (e.g., epithelial, connective, muscular, endocrine, intestinal, hematopoietic, nervous, bone, ligament, hepatic, cartilaginous, pancreatic, spleen, stomach, thymus, thyroid, dermal, esophageal, lung, adipose, ocular, ovarian, testicular or umbilical) or cells that can form a tissue of interest like adult (e.g., mesenchymal, hematopoietic or pancreatic), embryonic or fetal stem cells or progenitor cells can be used in the method to engineer the tissue.

Any of the aforementioned cells can be grown on the molded structure and, in one embodiment, the tissue engineered is an organ, and can be any organ capable of being engineered by one with skill in the art. In a particular embodiment, the cells that can be grown on the structure are neuronal cells and the tissue engineered a neural tube. These neural tubes can be used in the regeneration, repair or replacement of nervous tissue. Many different approches have been taken to restore nerve function after injury (e.g., laceration and/or severing of the nerve) including surgical repair, microsurgical nerve grafting, entubulation, nerve wrapping, fascicular tubulization and the use of various bioresorbable or non-resorbable conduits which serve to bridge nerve gaps (i.e., nerve guides). (See Li U.S. Pat. No. 4,963,146 and U.S. Pat No. 5,023,386 and the references cited therein, the contents of which are herein incorporated by reference). Unlike previous methods, a tissue can be engineered in the claimed method (i.e., a neural tube), that could replace a damaged nerve in addition to encourage the regeneration and integration of the engineered tissue with surrounding nerve tissue.

In a further embodiment of the method, a blood vessel is engineered by growing arterial/venous endothelial cells and/or vascular smooth muscle cells on a molded structure of the invention. As blood vessel damage is a cause of or complication in a number of diseases, there are many uses in the art for biocompatible artificial blood vessels. For instance, a blood vessel engineered in the method of the invention could be used to rectify blood vessel damage seen in most vascular conditions like cardiovascular diseases (e.g., atherosclerosis, coronary heart disease, hypertension) or cerebrovascular conditions (e.g., stroke, arteriolsclerosis or aneurysms), ataxia telangiectasia, hypertensive retinopathy, facial rosacea, varicose veins, polycystic kidney disease (characterized by high blood pressure and aneurysms), Behcet's Disease, liver cirrhosis, some connective tissue disorders (e.g, rheumatoid arthritis or Ehlers-Danlos syndrome), deep vein thrombosis, erythema, lupus and pulmonary edema.

Radially aligned channels in cylindrical and/or tubular scaffolds are conducive to cell migration along the radial path. In the case of a tubular structure, radially aligned channels can induce cell migration either into or out of a central lumen. Such a structure is thought to significantly improve the quality of regeneration of a variety of tissues. When a cell-impermeable membrane is added to such a structure on either the inner or outer surface of the tube wall, the cell-impermeable membrane prevents cell migration through that interface. Any cells observed in the tube wall after implantation will have migrated from the direction of the surface without the impermeable membrane.

Accordingly, in one embodiment of the method, the geometry of the mold is cylindrical and the porous structure is a tubular one having a cell-impermeable membrane on either the inner or outer surface of the tube wall. Thus, in a further embodiment, the migration of the cells grown on the tubular structure is controlled and/or prevented by the cell-impermeable membrane.

The present invention further relates to a porous molded structure that has radially aligned pore channels formed by a centrifugation technique. In a further embodiment, the porous molded structure is produced by the method of the invention, that is, by spinning a liquid suspension having two or more components of differing densities around a mold axis, immobilizing the two or more components to the mold and removing at least one of the two or more component phases from the bulk. The porous structures of the invention can be employed in a variety of applications in which a device or system has a porous or membranous structure, matrix or scaffold (e.g., separation, purification, filtration, concentration or cell growth/differentiation applications). Thus, in another embodiment of the method, the porous structure having radially aligned pore channels is used in tissue engineering, organ engineering, membranes for phase separation, dialysis tubes, water purification systems and liquid waste purification systems.

The present invention also relates to an engineered tissue or organ comprising a molded structure formed from a biocompatible material. The molded structure is seeded with cells along a radially aligned pore structure that is formed by a centrifugation technique. The tissue and/or organ can be any tissue or organ of interest capable of being engineered by the growth of a variety of cell types on the molded structure including neuronal cells, fibroblasts, epithelial cells, endothelial cells, epidermal cells, islets of Langerhan cells, osteocytes, tenocytes, chondrocytes, adult stem cells, embryonic stem cells, fetal stem cells or progenitor cells. In a particular embodiment, the molded structure is a tube that is seeded with neuronal cells such that the tissue engineered is a neural tube, primarily for use in nerve regeneration. In another embodiment, the arterial/venous endothelial cells and/or vascular smooth muscle cells are seeded on the radially aligned pore structure to produce a biocompatible blood vessel which can be used in a number of diseases characterized by blood vessel damage.

### EXEMPLIFICATION

### Example 1

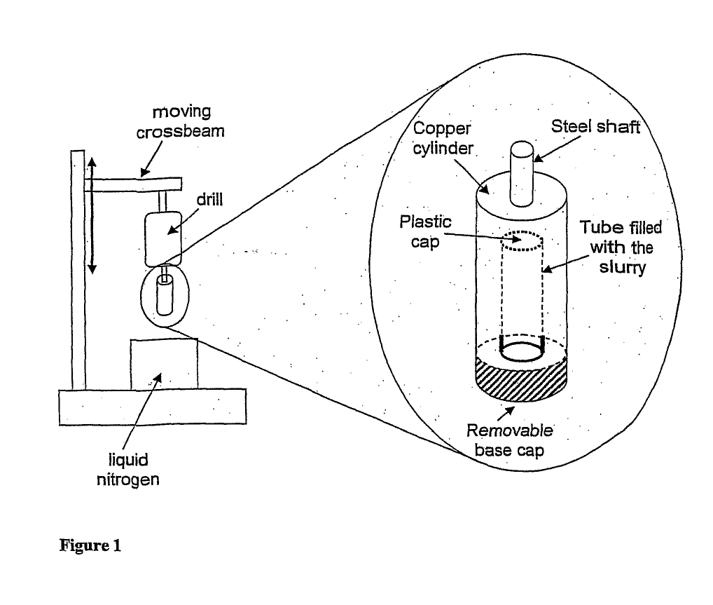

A suspension of type I collagen (0.5% w/v, acetic acid (0.05 M)) and chondroitin 6-sulfate (0.04% w/v) (a member of the glycosaminoglycan-GAG-family), was injected into a polyvinyl chloride (PVC) tube. The PVC tube was inserted into a hollow copper cylinder, with an internal diameter slightly larger ( 1/32″) than the external diameter of the PVC tube. The cooper mold (FIG. 1) was fabricated such that the bottom of the cylinder was threaded and could be sealed with a copper plug that was screwed into the tube; the top of the copper mold was sealed with a hardened steel rod.

The copper cylinder was held in place using the hardened steel plug in a mechanism able to spin the mold along its axis at a high rotational velocity (30,000 rpm) in air at room temperature for 15 minutes. During the spinning, the solid (collagen and GAG) and liquid (water and acetic acid) phases in the slurry underwent differential sedimentation along the cylinder radius; at the end of the spinning protocol, the solid phase was present in higher amounts in the external region of the cylindrical tube, while the liquid phase remained distributed throughout the mold but with more remaining around the central axis of the mold; the distribution was completely dependent on spin time and velocity.

After 15 minutes of continuous spinning, the cylinder was immersed in a liquid nitrogen bath while maintaining a constant rotational velocity for 2 minutes. The immersion in the liquid nitrogen bath freezes the liquid phase of the suspension in the distribution produced by spinning. Oxygen-free copper was used as the mold material due to its thermal properties, allowing for rapid heat conduction and freezing to facilitate rapid “locking” of the phase distribution.

The PVC tube filled with the frozen suspension was removed from the copper mold and the frozen liquid phase was removed by sublimation (Vacuum pressure: <100 mTorr; Temperature: 0° C.; Time 17 hours). A porous pattern, resembling the distribution of the sublimated liquid phase, remained inscribed in the solid dry structure. The solid phase, a porous tube, was removed from the PVC tube and underwent dehydrothermal treatment. The dehydrothermal treatment was used to induce physical crosslinks between the fibers of collagen in the tube to further stabilize the structure. The extent of the dehydrothermal treatment allowed for modulation of the degree of crosslinking, and thus the degradation rate of the tube, a critical adjustment necessary to optimize the bioactivity of the tube structure.

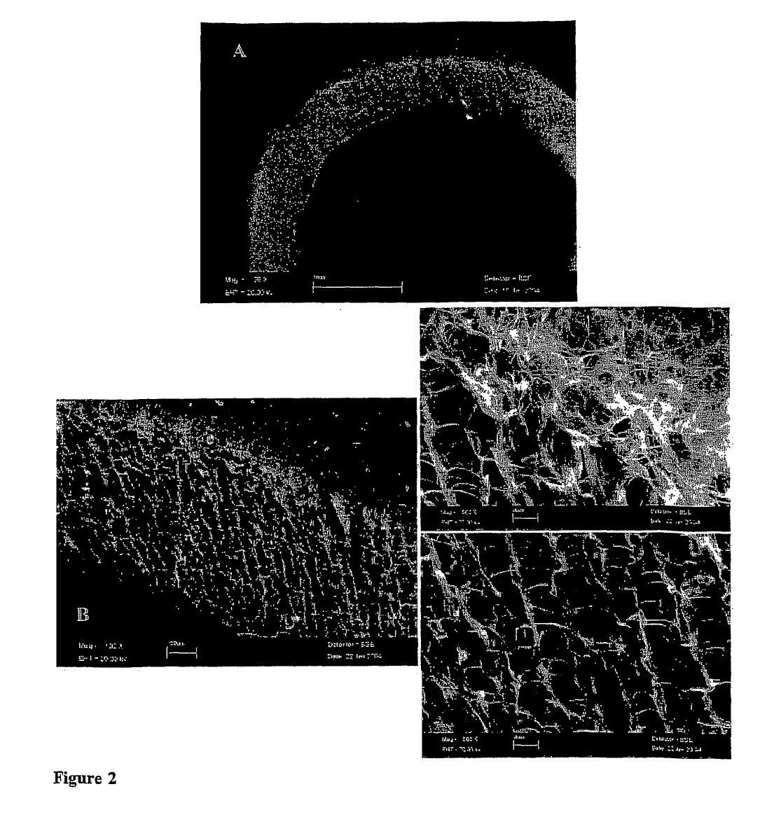

The tubular structure contained radially aligned pores extending from the internal lumen wall to the outer edge. At the outer edge of the tube wall, a significantly smaller average pore diameter and a significantly larger relative density was observed, consistent with the formation of a cell-impermeable membrane around the outside of the tube (FIG. 2).

### Example 2

The same collagen-GAG suspension as in Example 2 was used in a second experiment, where all conditions were kept constant except for the spinning velocity, which was changed from 30,000 rpm to 5,000 rpm, the spinning time prior to freezing, which was changed from 15 to 5 minutes, and the spinning time in liquid nitrogen, which was changed from 2 minutes to 1 minute.

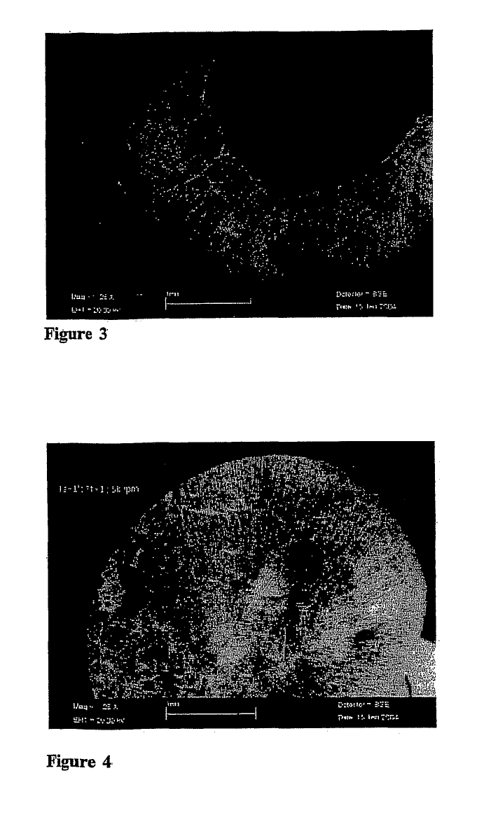

The external tube diameter of this tube remained constant compared to Example 1, while the internal diameter was considerably smaller due to the slower spinning velocity and reduced spinning time (smaller sedimentation effect). This tubular structure also displayed a radially aligned pore structure throughout the tube wall as well as a cell-impermeable membrane, although of reduced size, around the outer edge of the tube wall (FIG. 3).

### Example 3

The same procedure and the same collagen-GAG suspension was used as in Examples 2 and 3 only, in this case, the spinning time in air was changed from 5 minutes to 1 minute. The spinning velocity was 5,000 rpm and the spinning time in liquid nitrogen was 1 minute, as in Example 3.

Using this procedure, a porous cylinder was obtained; the sedimentation effect associated with the spinning was not great enough to create a complete separation between the solid and the liquid phase inside the cylinder, and thus the tube formation after freeze-drying. Instead, a complete cylinder of material was produced that maintained the radial alignment of pore structure observed in Examples 2 and 3 (FIG. 4).

While this invention has been particularly shown and described with references to preferred embodiments thereof, it will be understood by those skilled in the art that various changes in form and details may be made therein without departing from the scope of the invention encompassed by the appended claims.

1. A method for fabricating a porous molded structure comprising:
spinning a liquid suspension having two or more components of differing densities around a mold axis;
immobilizing the two or more components to the mold; and
removing at least one of the two or more component phases from the bulk, wherein radially aligned pore structures are formed in the molded structure.

2. The method of claim 1 wherein the mold geometry determines the shape of the outer surface of the porous molded structure.

3. The method of claim 2 wherein the mold geometry is cylindrical.

4. The method of claim 3 wherein one or more selected from the group consisting of: the density of the two or more components of the liquid suspension, the consistency of the liquid suspension, the spin time and spin velocity are modulated to produce the internal geometry of the porous cylindrical structure.

5. The method of claim 4 wherein the internal geometry comprises a central lumen that is selected from the group consisting of hollow and filled.

6. The method of claim 5 wherein the porous cylindrical structure is tubular.

7. The method of claim 6 wherein a cell-impermeable membrane is formed on a surface selected from the group consisting of the inner surface of the tube wall and the outer surface of the tube wall.

8. The method of claim 7 wherein the two or more components are immobilized to the mold by immersion of the bulk in liquid nitrogen.

9. The method of claim 8 wherein one or more selected from the group consisting of: the density of the two or more components of the liquid suspension, the consistency of the liquid suspension, the spin time, spin velocity and the immobilization technique are modulated to produce the internal pore structure of the porous molded structure.

10. The method of claim 9 wherein at least one of the two or more component phases are removed from the mold by a process selected from the group consisting of: lyophilization, leaching using a diffusion gradient, enzymatic digestion, phase inversion and thermal degradation.

11. The method of claim 10 further comprising further stabilizing the porous structure.

12. The method of claim 11 wherein one of the two or more components of the liquid suspension is collagen.

13. The method of claim 12 wherein the porous structure is further stabilized using a dehydrothermal treatment to induce physical crosslinks of the collagen fibers.

14. A method for engineering a tissue comprising:
fabricating a porous molded structure having radially aligned pore channels, and
growing cells on the porous molded structure along the radially aligned pore channels.

15. The method of claim 14 wherein the geometry of the molded structure is cylindrical.

16. The method of claim 15 wherein the cells grown on the radially aligned pore structure of the molded structure are neuronal cells, fibroblasts, epithelial cells, endothelial cells, epidermal cells, islets of Langerhan cells, osteocytes, tenocytes, chondrocytes, adult stem cells, embryonic stem cells, fetal stem cells or progenitor cells.

17. The method of claim 16 wherein the tissue engineered is an organ.

18. The method of claim 17 wherein the porous cylindrical structure is tubular.

19. The method of claim 18 wherein the porous tubular structure has a cell-impermeable membrane on a surface selected from the group consisting of the inner surface of the tube wall and the outer surface of the tube wall.

20. The method of claim 19 wherein the migration of the cells grown on the tubular structure is prevented by the cell-impermeable membrane.

21. The method of claim 20 wherein the cells seeded on the radially aligned pore structure of the molded structure are neuronal cells and the tissue engineered is a neural tube.

22. The method of claim 21 wherein the cells grown on the radially aligned pore structure of the molded structure are arterial endothelial cells or venous endothelial cells and vascular smooth muscle cells and the tissue engineered is a blood vessel.

23. A porous molded structure comprising radially aligned pore channels formed by a centrifugation technique.

24. The porous molded structure of claim 23 wherein the structure is produced by the method of claim 1.

25. The porous molded structure of claim 24, wherein the structure is cylindrical.

26. Use of the porous cylindrical structure of claim 25 in one or more applications selected from the group consisting of tissue engineering, organ engineering, membranes for phase separation, dialysis tubes, water purification systems and liquid waste purification systems.

27. An engineered tissue or organ comprising a molded structure formed from a biocompatible material, said molded structure seeded with cells grown along a radially aligned pore structure formed by a centrifugation technique.

28. The engineered tissue of claim 27 wherein the cells seeded on the radially aligned pore structure of the molded structure are cells selected from the group consisting of neuronal cells, fibroblasts, epithelial cells, endothelial cells, epidermal cells, islets of Langerhan cells, osteocytes, tenocytes, chondrocytes, adult stem cells, embryonic stem cells, fetal stem cells and progenitor cells.

29. The engineered tissue of claim 28 wherein the molded structure is tubular.

30. The method of claim 29 wherein the cells seeded on the radially aligned pore structure of the molded structure are neuronal cells and the tissue engineered is a neural tube.

31. The method of claim 29 wherein the cells seeded on the radially aligned pore structure of the molded structure are arterial endothelial cells or venous endothelial cells and vascular smooth muscle cells and the tissue engineered is a blood vessel.
